(19) United States
(12) Patent Application Publication
(10) Pub. No.: US 2017/0258546 A1
(43) Pub. Date: Sep. 14, 2017

(54) SURGICAL TRAY SYSTEM

(71) Applicant: Krishnan K. Ghosh, Chicago, IL (US)

(72) Inventor: Krishnan K. Ghosh, Chicago, IL (US)

(21) Appl. No.: 15/606,305

(22) Filed: May 26, 2017

Related U.S. Application Data
(63) Continuation of application No. 14/964,441, filed on Dec. 9, 2015.
Application No. 14/964,441 is a continuation of application No. 13/678,843, filed on Nov. 16, 2012, now abandoned.

(51) Int. Cl. A61B 50/33; A61B 46/10; A61B 90/00; A61B 90/30; A61B 50/00; A61B 50/30; A61B 90/50

(52) U.S. Cl. CPC A61B 50/33; A61B 2050/005; A61B 2050/3008; A61B 2050/3015; A61B 2050/314; A61B 2090/3616; A61B 90/50; A61B 90/30; A61B 90/361; A61B 46/10

(57) ABSTRACT

A surgical tray system is disclosed. The system includes a tray for use in an operating room environment. In various embodiments, the tray includes an indentation having a bowl shape. A vacuum sealed bag is fitted over the tray and form fitted to the tray when the air is removed from within the bag. The tray optionally includes an light source for illuminating an area under the tray, a magnification element, and pouches for storing items used during a surgical procedure. The tray is optionally substantially transparent to allow light to pass through the tray. The tray is ideally positioned over a patient bed. It may be mounted via an arm to a bed frame member, a monitor station, or a ceiling fixture.

### CROSS REFERENCE

This application is a continuation of U.S. application Ser. No. 14/964,441, filed on Dec. 9, 2015, which is a continuation of U.S. application Ser. No. 13/678,843, filed on Nov. 16, 2012, the entire contents of both of the aforementioned applications are incorporated by reference herein.

### BACKGROUND OF THE INVENTION

Field of the Invention

The present invention relates to surgical tray systems, and more particularly, to a surgical tray having various features for use in an operating room environment.

Background

A conventional operating room setup typically includes various fixtures including a back table. A back table is a sterilized table which is used to hold instrumentation needed during a surgical procedure. In order to maintain a sterile environment, the table must either be completely sterilized, covered, or disposable. It is typically located laterally separated from the patient operating bed. Because of this arrangement, it can be inconvenient for a surgeon to move between the patient bed and the back table. Furthermore, items may be dropped and compromised when they are being moved from the back table to the patient bed.

Typical back tables also require various items and configurations which are selected based on the procedure being performed and the preferences of the surgeon. Often, a bowl with sterile fluid is used. This bowl typically must be disposed of after use, and can be susceptible to spilling. Mayo Stand type tables have been proposed for use to allow a tray to overhang a bed, however, various limitations make the use of such devices inconvenient, as described below.

A number of conventional back tables or stands have been proposed and used in the industry, however, they lack various helpful configurations. For example, U.S. Pat. No. 3,738,405 issued to Ericson discloses a Mayo Stand with a cover. The stand can be moved over a bed, however, its configuration results in a very limited range of positioning options, and it can block light and be in the way of the surgeon if it were used at an operating bed. Any containers or bowls would have to be disposable and loosely placed above the stand, being susceptible to being spilled.

U.S. Pat. No. 5,170,804 issued to Glassman discloses a Mayo Stand with a disposable drape. The drape can hold a kit of instruments which can be laid out on the stand. However, the same limitations with positioning, blocking light, and obstructing access to the patient apply to this design as well.

Another Mayo Stand cover is disclosed by U.S. Pat. No. 5,411,036 issued to Wilkes. Configurations such as these address the issue of covering the stand, however, the cover is inconvenient to apply, and it does not address the various issues raised above. The various convention cover or drape systems give rise to numerous problems, including drapes not conforming closely to the underlying tables, and having the risk of sliding off of surfaces, which can compromise sterility of surfaces and supplies.

U.S. Pat. No. 5,435,322 issued to Marshall discloses a ring stand basin liner. This allows a basin to be supported by a stand, and a sterile liner is used to line the basin so that the basin need not be sterilized between uses. However, there is no room for any further tools or supplies to be placed on the stand, and it still must be located separate from the patient bed.

Another Mayo Stand cover is disclosed in U.S. Pat. No. 5,871,015 issued to Lofgren et al., and a further surgical table cover is disclosed in U.S. Pat. No. 7,104,201 issued to Comeaux et al. Other cover systems and Mayo Stands exist, however, none of these conventional devices overcome the problems of bowls being spilled, requiring bowls to be disposed of, limitations in range of positioning, light blockage, etc.

In U.S. Pat. No. 6,142,152 issued to Gawarecki, a cover for an operating room back table is disclosed. This cover unwraps a kit of supplies and covers the stand. An air permeable section of the cover helps enable the cover to be unwrapped quickly. The same limitations as discussed above apply.

Surgeons often find it convenient to have compartments available where items can be placed or stored during a procedure. U.S. Pat. No. 6,874,505 issued to Fenwick et al. discloses a surgical drape system which covers the patient. The drape includes a pouch which can be accessed during the procedure. However, this is highly inconvenient for a number of procedures as portions of the patient's body may be blocked from access. Furthermore, this drape clearly does not replace the need for a tray or table top for further supplies.

None of these prior devices provide a surgical tray surface which can be conveniently located, moved with positioning limitations, used without blocking light, or which eliminates problems with free-standing bowls, among other issues. Thus, there continues to be a need for a practical, versatile surgical tray system which allows for convenient use and meets a variety of needs.

### SUMMARY

The present invention is a surgical tray system for use in an operating room environment. The tray system addresses a range of needs in a convenient manner. The tray system of the present invention is intended to either replace the conventional back table in an operating room environment, or to be used in addition to such a back table.

In various embodiments, the surgical tray system of the present invention is covered with a sterile vacuum sealed covering element. The covering element may conveniently be a vacuum sealed bag having a valve which allows air to be suctioned out of the bag to form fit the bag to the tray. Air can be vacuumed out of the bag using any suitable vacuum system, however, it may be most convenient to use the vacuum system and tubing which is typically present in most operating room environments. The bag has an opening which allows it to be placed over the tray. This opening can be sealed in any suitable manner. For example, it may be sealed using a zip lock type of closure, a dual zip lock type of closure, an adhesive closure such as a peel away covered adhesive strip, or by any other suitable method. Optionally, to facilitate air removal, various holes may be formed through the tray so that air pockets do not become formed in the bag which cannot be suctioned out, if necessary. Many configurations are possible. For example, holes may be formed to pass all the way through the tray, or merely from the upper surface to an interior cavity of the tray, and air might be suctioned out a side hole in the cavity.

The bag can be removed, ideally be inverting it so that disposables can be wrapped within the bag upon removal of the bag from the tray system. Once inverted, the bag can be sealed in inverted condition, and the air can be vacuumed out of the bag either through the same valve, a dual valve, or a secondary valve, thus reducing the volume of the waste. The tray is thus reusable and need not be sterilized after each use. Furthermore, this vacuum cover system enables the number of disposables to be reduced, the risk of spillage of supplies or containers to be reduced, and it reduces the footprint of disposables after a procedure. It also reduces the need for time and resources used in sterilizing supplies such as containers which, by the present invention, may be covered with the sterile vacuum sealed bag.

In various preferred embodiments, the tray system can be attached to an arm which mounts to a portion of the patient bed frame. The arm is configured to allow motion in any desired direction, and stability once positioned. A safety stop prevents the tray system from being lowered too far down toward the patient.

In other embodiments, the tray system may be mounted to a monitor system typically used in an operating room environment, or to a ceiling fixture. In any of these configurations, the tray ideally can be freely moved and positioned as desired. The mounting arm in any of these configurations may be covered with a sterile bonnet when assembled. It may attach to the tray system via a clamp configuration, or any other suitable mounting method.

In certain preferred embodiments, the tray system includes an integral indentation in an upper surface so as to form a container or bowl shape. This bowl is covered with the vacuum bag, and the vacuum bag form fits into the bowl when the air is suctioned out of the bag. Thus, a sterile bowl is formed which can hold fluid without the risk of spilling the bowl, and without the need to dispose of or sterilize the bowl after use. Other shapes or containers may also be molded or formed in the tray system, thereby eliminating the need for additional instruments or containers which must either be sterilized after each use or disposable.

In some embodiments, the tray system is substantially transparent. This allows light to pass through the tray so that areas below the tray which need to be illuminated during a procedure are not blocked by the tray. Furthermore, this provides for visibility through the tray when necessary.

Certain preferred embodiments also include a magnification element. This may be implemented by forming a magnifying lens as part of the tray itself which allows a user to be able to see a magnified image through the tray. This can be useful or necessary during certain procedures. Alternatively, the magnifying element may be a separate piece which attaches to the tray at a side edge. In certain embodiments, attachment elements are included around the perimeter of the tray to enable other items to be added to the configuration.

In various other embodiments, a light source is included positioned to illuminate an area underneath the tray. For example, an LED component may be positioned on the underside of the tray. Thus, an area of a patient may be illuminated during a procedure. Optionally, the light source may be selectively positioned to illuminate specific areas as needed.

In other embodiments, pouches may be provided on the exterior of the bag at or below the edge of the tray. Such pouches may be integral with the bag, or may be adapted to be added to the exterior of the bag. This allows for various items to be stored conveniently in the pouches during a procedure. Labels can be used to identify the contents of the pouches.

In certain embodiments, the bag of the system can be stored before use in a sealed outer bag, and can be wrapped up, along with optional supplies, and placed in an outer shell. This enables compact storage. The outer shell may be provided to meet various other needs. For example, in some embodiments, a portion of the outer shell may be placed on the patient bed and have a passageway through which a tool such as a wire for use in angioplasty can be placed. This will hold it in place to prevent it from accidentally touching a non-sterile surface such as the floor.

Still other objects and advantages of the present invention will become readily apparent to those skilled in the art from the following detailed description, wherein it is shown and described only the preferred embodiments of the invention, simply by way of illustration of the best mode contemplated of carrying out the invention. As will be realized, the invention is capable of other and different embodiments and its several details are capable of modifications in various obvious respects, without departing from the invention. Accordingly, the drawings, wherein like reference numerals represent like features, and description are to be regarded as illustrative in nature and not as restrictive.

### BRIEF DESCRIPTION OF THE DRAWINGS

FIG. 1 shows a perspective view of one embodiment of the tray system of the present invention.

FIG. 2 shows a perspective view of another embodiment of the tray system of the present invention.

FIG. 3 shows a perspective view of another embodiment of the tray system of the present invention.

FIG. 4 shows a perspective view of another embodiment of the tray system of the present invention.

FIG. 5 shows a perspective view of another embodiment of the tray system of the present invention.

FIG. 6 shows a perspective view of another embodiment of the tray system of the present invention.

FIG. 7 shows a perspective view of one embodiment of a container for the vacuum sealed bag and other items according to the present invention.

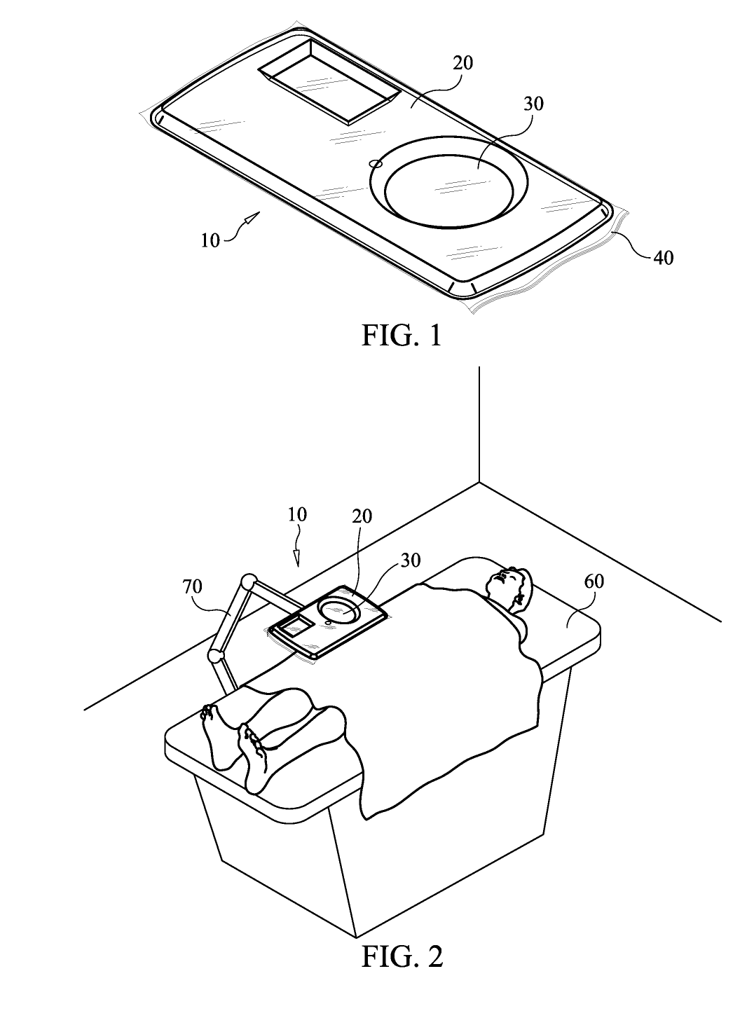

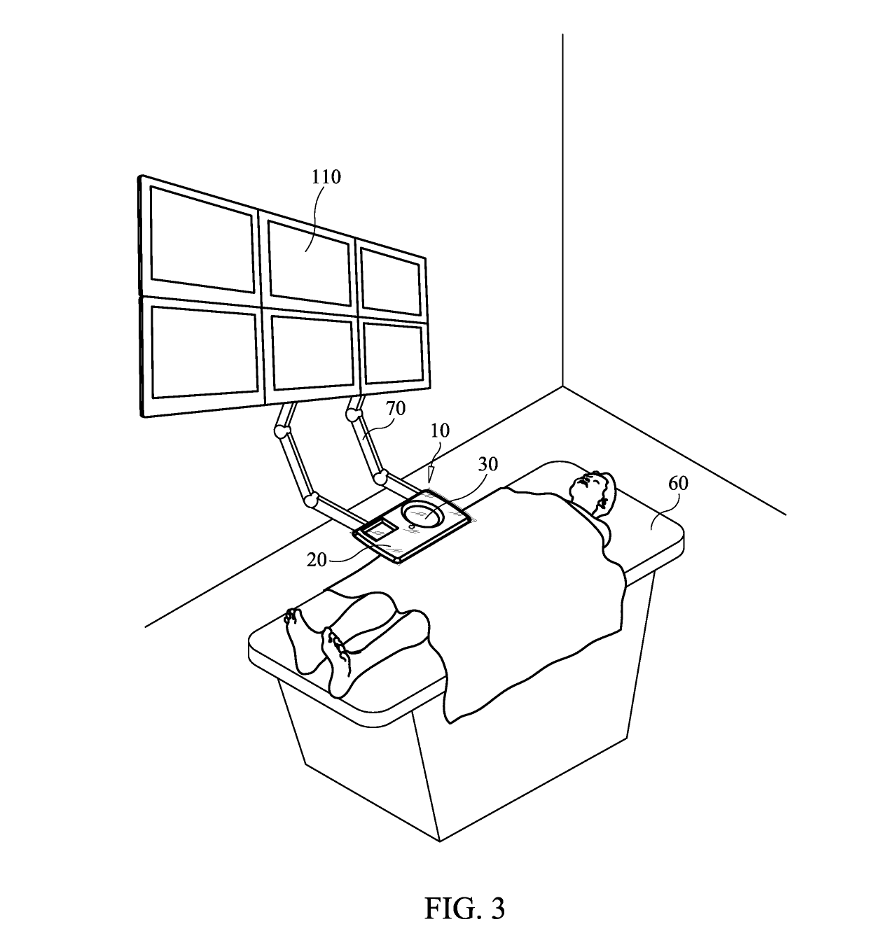

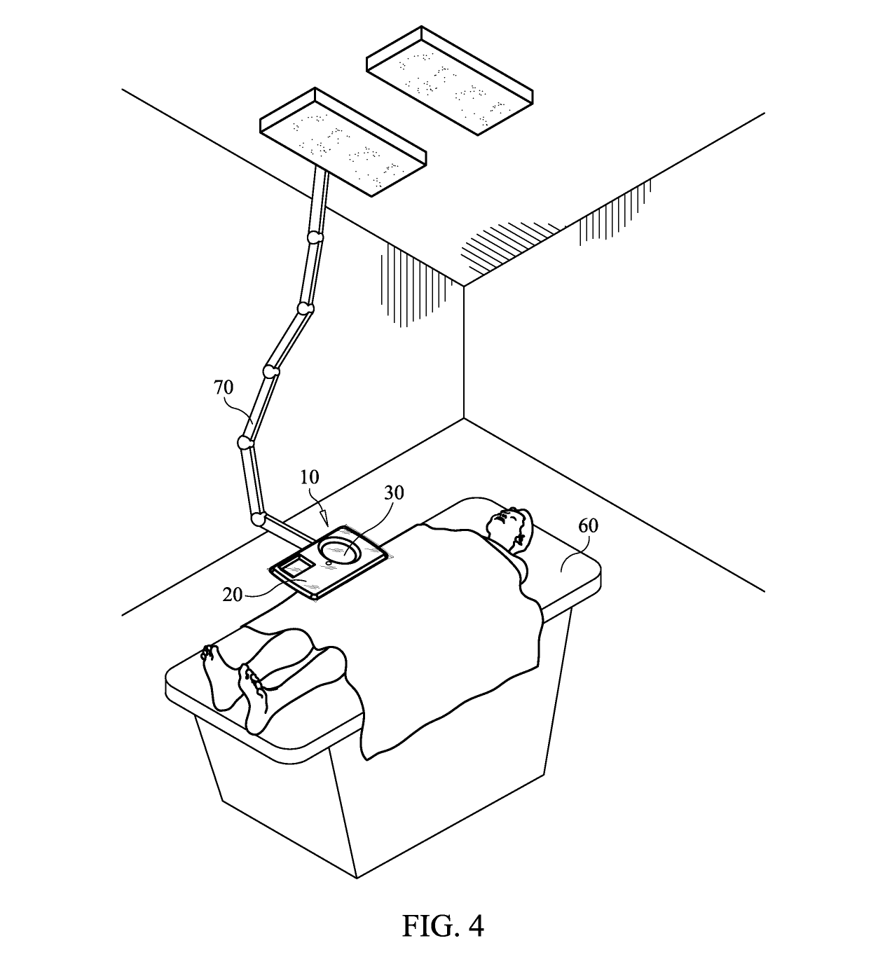

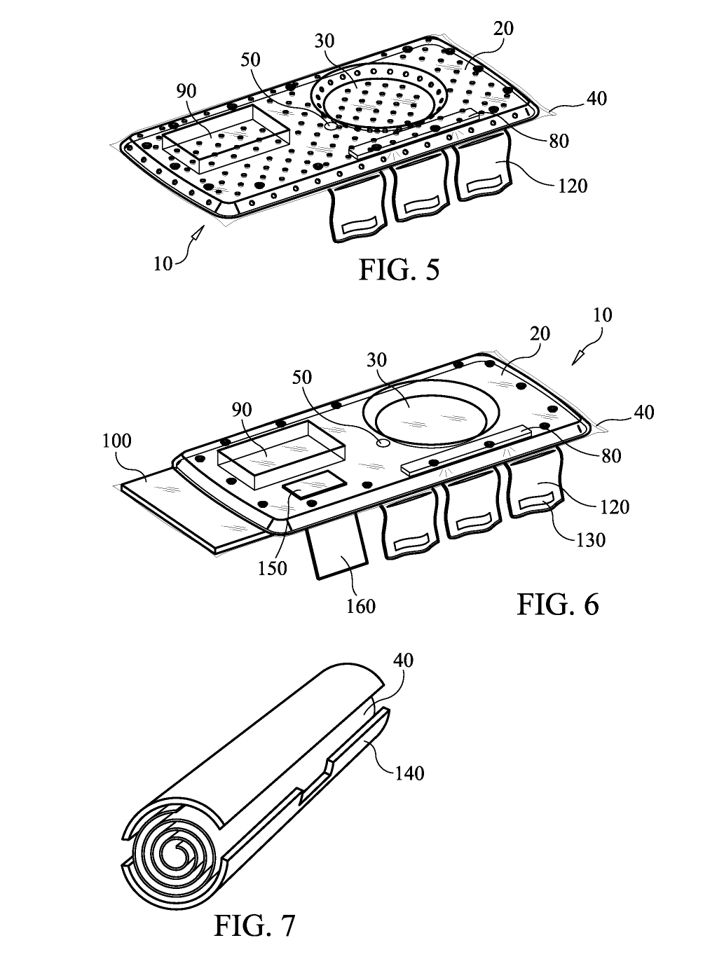

### DETAILED DESCRIPTION OF THE INVENTION

While this invention is susceptible of embodiments in many different forms, there are shown in the drawings and will herein be described in detail, preferred embodiments of the invention with the understanding that the present disclosure is to be considered as an exemplification of the principles of the invention and is not intended to limit the broad aspect of the invention to the embodiments illustrated.

The present invention is a surgical tray system 10. The system 10 includes a substantially planar tray 20 having an upper surface and a lower surface. In various embodiments, the tray 20 includes an indentation 30 formed in the upper surface of the tray 20. The indentation 30 may take the shape of a container or a bowl. It is contemplated to be within the scope of the present invention that additional indentations taking on any desired shape for other purposes may be added.

In certain preferred embodiments, the tray 20 is covered by a vacuum fitted covering element 40 which covers at least the upper surface of the tray 20 and becomes form-fitted into the indentation 30 when air is removed from the bag. Ideally, the bag includes a valve 50 through which air can be removed.

In various embodiments, the surgical tray system 10 configured and adapted to be movably mounted above a patient bed 60. For example, the tray 20 may be mounted to an arm 70 which is attached to a bed frame member. Ideally, an articulating arm 70 is used that is configured to allow a range of positioning options. Thus, the tray 20 can be moved side to side, front to back, and up and down. Ideally, a limiting safety feature is included to prevent the tray 20 from moving too far down toward the patient. The arm 70 may be jointed in various manners to enable this full range of movement.

In other embodiments, a similar arm 70 may mount the tray system 10 to a monitor system 110 such as those typically used in an operating room, or to a ceiling fixture. The arm 70 would allow the tray 20 to be positioned in a range of suitable positions.

The tray 20 can be formed of a plastic resin, molded, or formed of any suitable material. In some embodiments, the tray 20 is substantially transparent. This allows light to pass through the tray 20 so that it does not block light which is needed to illuminate an area during a procedure. Furthermore, this allows a surgeon to be able to see through the tray 20 when necessary.

In various embodiments, the tray system 10 may also include a light source 80 adapted and positioned to illuminate an area beneath the tray 20. The light source 80 may be any suitable light source, such as an LED light source. It may optionally be positionable so that a user can direct it to a desired area.

In particular embodiments, the tray system 10 includes a magnification element 90 or 100 positioned and adapted to magnify an area below the lower surface of the tray 20 when viewed from above the tray 20. FIG. 6 illustrates various alternate manners of providing a magnification element. An integral magnification element 90 may be formed in the tray 20. Alternatively, an attachable magnification element 100 may be attached to an edge of the tray 20. Thus, a surgeon, for example, can use this for magnification when needed during a procedure. Various other alternative magnification elements are also contemplated. For example, a magnification element 150 may be integral with the vacuum bag 40 itself, or may be an extension of the bag 40 such as a flap 160, which may either be integral with the bag 40 or affixed to the exterior of the bag 40.

In various preferred embodiments, external pouches 120 are provided and adapted to provide a storage area mounted externally on the bag 40. Such pouches 120 may be formed integrally with the bag 40, or may be attached to the external portion of the bag 40 during use. These pouches provide a storage area, and may be labeled using stickers 130, for example.

The vacuum bag 40 of the present invention may stored in a sterilized package, such as an outer sleeve or bag, and rolled up and placed in a shell 140. This shell 140, or a portion thereof, is ideally configured to provide a useful channel. The shell 140 can be placed on a patient bed during a procedure such as an angioplasty procedure, and a tool such as the wire used for such a procedure, may be conveniently positioned within this channel to prevent it from accidentally being placed in contact with a non-sterile surface such as the floor.

While specific embodiments have been illustrated and described, numerous modifications come to mind without significantly departing from the spirit of the invention and the scope of protection is limited by the scope of the accompanying claims.

1. A surgical tray system comprising:
a substantially planar tray having an upper surface and a lower surface,
an indentation formed in said upper surface
a vacuum fitted covering element covering at least said upper surface and form-fitted into said indentation.

2. The surgical tray system according to claim 1, wherein said tray table is configured and adapted to be movably mounted above a patient bed.

3. A surgical tray system comprising:
a substantially planar tray having an upper surface and a lower surface,
a vacuum fitted covering element having an opening adapted to allow air to be vacuumed through covering at least said upper surface,
wherein said tray table is configured and adapted to be movably mounted above a patient bed.

4. The surgical tray system according to claim 3, wherein at least a portion of said tray is substantially transparent.

5. The surgical tray system according to claim 3, further comprising a light source mounted to said tray and adapted and positioned to illuminate an area beneath said tray.

6. The surgical tray system according to claim 3, wherein said tray further comprises an indentation formed in said upper surface, wherein said vacuum fitted covering is substantially form-fitted into said indentation.

7. The surgical tray system according to claim 3, further comprising a magnification element positioned and adapted to magnify an area below the lower surface of said tray when viewed from above said tray.

8. The surgical tray system according to claim 3, further comprising at least one pouch adapted to provide a storage area on the external portion of said bag.

9. The surgical tray system according to claim 3, wherein said tray is mounted to a frame member of a patient bed via a movable arm.

10. The surgical tray system according to claim 3, wherein said tray is mounted to a monitor station via a movable arm and positioned above a patient bed.

11. The surgical tray system according to claim 3, wherein said tray is mounted to a ceiling fixture via a movable arm and positioned above a patient bed.

12. A surgical tray system comprising:
a substantially planar tray having an upper surface and a lower surface,
said tray being substantially transparent,
wherein said tray table is configured and adapted to be movably mounted above a patient bed.

13. The surgical tray system according to claim 12, further comprising a vacuum fitted covering element having an opening adapted to allow air to be vacuumed through covering at least said upper surface.

14. The surgical tray system according to claim 12, further comprising a magnification element positioned and adapted to magnify an area below the lower surface of said tray when viewed from above said tray.

15. The surgical tray system according to claim 12, further comprising a light source mounted to said tray and adapted and positioned to illuminate an area beneath said tray.

16. The surgical tray system according to claim 13, wherein said tray further comprises an indentation formed in said upper surface, wherein said vacuum fitted covering is substantially form-fitted into said indentation.

17. A surgical tray system comprising:
a substantially planar tray having an upper surface and a lower surface,
said tray system comprising a magnification element adapted to allow an image to be magnified therethrough,
wherein said tray table is configured and adapted to be movably mounted above a patient bed.

18. The surgical tray system according to claim 17, wherein said magnification element is integral with a vacuum bag which envelopes at least a portion of said tray.

19. A surgical tray system comprising:
a substantially planar tray having an upper surface and a lower surface,
said tray comprising an illumination element adapted to illuminate an area below said tray,
wherein said tray table is configured and adapted to be movably mounted above a patient bed.

20. The surgical tray system according to claim 19, further comprising a vacuum fitted covering element having an opening adapted to allow air to be vacuumed through covering at least said upper surface.

21. The surgical tray system according to claim 20, wherein said tray further comprises an indentation formed in said upper surface, wherein said vacuum fitted covering is substantially form-fitted into said indentation.
